(19) United States
(12) Patent Application Publication
(10) Pub. No.: US 2014/0309942 A1
(43) Pub. Date: Oct. 16, 2014

(54) POLLUTION MONITORING

(71) Applicant: Odd Ketil Anderson, Stavanger (NO)

(72) Inventor: Odd Ketil Anderson, Stavanger (NO)

(73) Assignee: BIOTATOOLS AS, Stanger (NO)

(21) Appl. No.: 14/238,162

(22) PCT Filed: Aug. 10, 2012
(86) PCT No.: PCT/GB2012/051956

§ 371 Date: Jun. 6, 2014

(30) Foreign Application Priority Data
Aug. 10, 2011 (GB) 1113784.1

(51) Int. Cl. G01N 33/50; G06F 19/24

(52) U.S. Cl. CPC G01N 33/5085; G06F 19/24
USPC 702/19

(57) ABSTRACT

The effect of pollution in an aquatic mass is monitored. A living sessile organism (2) is placed in said aquatic mass and a series of first measurements of a first parameter of said organism (2) is carried out during a first time period. Each of said first measurements is recorded with an associated measurement of a second parameter to give a series of first data points (82c). A first envelope (80c) is determined and recorded with respect to said first parameter for said first data points (82c). A series of second measurements of said first parameter of said organism (2) is carried out during a second time period. Each of said second measurements is recorded with an associated measurement of a second parameter to give a series of second data points (86c). A second envelope (84c) is determined and recorded with respect to said first parameter for said second data points (86c). Said first and second envelopes (80c, 84c) are compared to determine the presence of pollution during said first or second time period. The first and second envelopes (80c, 84c) may be lower envelopes.

This invention relates to improvements in and relating to methods of monitoring pollution in an aquatic mass, and to software for use in such methods.

Pollution of aquatic masses, e.g. oceans, seas, lakes and rivers, may arise through an accidental spill, or as a consequence of a deliberate discharge either of which take the form of the release into the aquatic mass of chemicals which affect the ability of the indigenous flora and fauna to thrive. In the case of deliberate discharges, these may be legally permitted but nonetheless eventually prove unexpectedly to be harmful. In the case of fixed installations which may be liable to be alleged to be a pollution source, as well as for operators of fixed installations which may suffer detrimental effects from aquatic pollution, it is desirable for the aquatic mass to be monitored to detect pollution events in order that compensatory or protective action may be taken or in order to demonstrate that in fact legal compliance has been achieved. Such installations will typically comprise offshore drilling or hydrocarbon recovery installations, ports and other land/water material transfer locations, land-based industrial, municipal, and private discharges, fish farms and the like. Pollution detection can also be helpful in identifying previously unknown side-effects of legal discharges.

Many multi-cellular non-mammalian aquatic animals, e.g. fish, shellfish, etc. exhibit detectable changes in physiology/behaviour in response to exposure to pollution which are far more sensitive and relevant than simply measuring death. Such behaviour includes alteration of growth rate, alteration of heart beat and alteration in shell opening and closing behaviour. The use of such animals, so-called “indicator” or “sentinel” species, in real time environmental monitoring (RTEM) methods is widely known and is described for example in WO 2007/086754 and WO 2009/013503, the contents of which are incorporated herein by reference. An important advantage of RTEM methods is that they are non-invasive.

Two systems have evolved which have been used on marine organisms, one based on physiological responses (heart rate monitoring), the other based on behaviour (valve gaping in mussels). However, such systems are most responsive to acute incidents rather than long term, low dose responses. There still exists a need to develop RTEM methods that can directly measure important parameters sensitive to low dose chronic exposure situations used in environmental risk assessment and management and which will reduce the use of invasive methods as biomarkers in environmental monitoring. The present invention seeks to address this need and, in particular, to provide alternative methods of directly monitoring aquatic animals which can be used to monitor pollution both in the short and longer term.

The Applicant has appreciated that environmental impact can be assessed effectively by examining the envelope of measurements of pollution-sensitive parameters against particle concentration and/or temperature and specifically changes therein over time. Examples of such pollution-sensitive parameters are particle clearance rate (net intake of particles, which includes food particles, by the organism), pumping rate (the rate at which ambient water passes through the organism), growth rate of the organism and oxygen consumption rate (which indicates metabolic activity) of the organism. This recognises that whilst pollution-sensitive parameters may be affected by other factors (e.g. clearance rate may be affected by the degree of valve gape), the maximum and minimum measurements of various parameters respectively are dependent on the level of pollution. Specifically the Applicant has realised that the effect of pollution is that for a given particle concentration or temperature the minimum measurements observed across a series of measurements of certain parameters (e.g. oxygen concentration in a region adjacent an excretion orifice of an organism) are reduced, and of certain other parameters (e.g. particle number or chlorophyll a fluorescence in a region adjacent an excretion orifice of an organism) are increased, as compared to when there is no, or less, pollution present. Further, the Applicant has realised that the effect of pollution is that for a given particle concentration or temperature the maximum measurements observed across a series of measurements are reduced (e.g. particle clearance) or increased (e.g. oxygen consumption) as compared to when there is no, or less, pollution present.

When viewed from a first aspect the invention provides a method of monitoring the effect of pollution in an aquatic mass comprising the steps of:
placing a living sessile organism in said aquatic mass;
carrying out a series of first measurements of a first parameter of said organism during a first time period;
recording each of said first measurements with an associated measurement of a second parameter to give a series of first data points;
determining and recording a first envelope with respect to said first parameter for said first data points;
carrying out a series of second measurements of said first parameter of said organism during a second time period;
recording each of said second measurements with an associated measurement of a second parameter to give a series of second data points;
determining and recording a second envelope with respect to said first parameter for said second data points; and
comparing said first and second envelopes to determine the presence of pollution during said first or second time period.

The invention extends to software, and a carrier bearing software, comprising instructions for configuring a processor to carry out the steps of:
receiving an input of a series of first measurements of a first parameter of an organism during a first time period;
receiving an input of an associated measurement of a second parameter for each of said first measurements;
recording each of said first measurements with the associated measurement of the second parameter to give a series of first data points;
determining and recording a first envelope with respect to said first parameter for said first data points;
receiving an input of a series of second measurements of said first parameter of said organism during a second time period;
receiving an input of an associated measurement of the second parameter for each of said first measurements;
recording each of said second measurements with the associated measurement of the second parameter to give a series of second data points;
determining and recording a second envelope with respect to said first parameter for said second data points; and
comparing said first and second envelopes to determine the presence of pollution during said first or second time period.

The first and second envelopes may be upper envelopes. In a preferred set of embodiments the first and second envelopes are lower envelopes.

The second parameter may be particle concentration, temperature or time or any other suitable parameter. The first and second data points could be two-dimensional—i.e. comprising one second parameter, three dimensional—i.e. comprising two parameters other than the first parameter, or higher dimensional. In the case of two-dimensional data points the envelope is a line, whereas in the case of three-dimensional data points the envelope is a surface.

If pollution is determined this may cause an alarm or alert to be triggered. Pollution is indicated by a change in the upper or lower envelope such that it is increased or decreased. This could occur in either the first or the second time period—i.e. the method could be used to detect to appearance or disappearance of pollution. In some embodiments a quantitative measure of pollution may be determined through a known or empirically-established relationship with the amount by which the upper envelope or the lower envelope is increased or decreased.

In a set of embodiments the first parameter is a particle clearance rate. In some preferred embodiments, the method comprises the steps of:
performing a series of first measurements, of a flux of particles in said aquatic mass in a region adjacent a feeding orifice of the organism;
performing a series of second measurements, of a flux of particles in said aquatic mass in a region adjacent an excretion orifice of said organism; and
using said first and second measurements to calculate a particle clearance rate for said organism.

The particle clearance rate (i.e. the net intake of particles) for a given organism is the difference between its total intake of particles and the outflow of particles therefrom, and accordingly the outlet particle flux or outlet particle density (where ‘outlet’ indicates that the parameter is measured in a region adjacent an excretion orifice of the organism) can be used as an indicator of pollution level. In practice the particle clearance rate varies with degree of gaping of the organism such that the maximum measurements across a series of measurements of the particle clearance rate depend on the pollution level. Correspondingly, the outlet particle density and outlet particle flux vary with degree of gaping so that the minimum measurements across a series of measurements depend on the pollution level. In this case, the change in pollution level is determined from the change in a lower envelope associated with the outlet particle density or outlet particle flux data.

In a set of embodiments, the first parameter is a particle flux, or a particle density, measured in a region adjacent an excretion orifice of the organism. The particle flux or particle density may be expressed as a percentage or fraction of the corresponding value in ambient water or of the corresponding value in a region adjacent a feeding orifice of the organism.

The particle clearance rate (or correspondingly, the outlet particle density or outlet particle flux) of a sessile organism is used as a non-invasive, in-situ indicator of pollution since the applicant has appreciated that the particle clearance rate is a strong indicator of the organism's scope for growth, which in turn is a good indicator of aquatic pollution. Scope for growth (SFG) is the energy budget of an organism calculated by measuring food uptake (clearance rate), excretion (faecal production) and oxygen consumption. However it has been found that the scope for growth is dominated by the clearance rate (e.g. of the order of: clearance rate 50-60%, faecal production 10-20% and oxygen consumption 15-25%). It is feasible therefore to measure just the clearance rate in place of SFG. The SFG or clearance rate is a strong and sensitive method of showing the effect of pollution directly in an aquatic mass.

The flux of particles could simply be defined as the number of particles passing through a given planar area in a given time, but in preferred embodiments the size distribution is also measured. By taking the size distribution into account an estimate of the mass and/or volume flux can be made which gives a more accurate picture of the potential food value. By examining the fluxes measured adjacent the intake of the organism and adjacent its outflow respectively, the uptake of food by the organism, i.e. the clearance rate can be calculated.

It follows from the above that the particle clearance rate calculated in accordance with the invention could take a number of forms. It could be the number of particles retained by the organism in a given time or from a given volume of water in which the particles are suspended that passes through the organism. Alternatively it could be the aggregate size or volume of particles retained in a given time. However there are other possibilities such as the number of particles over a predetermined size or within a predetermined range of sizes that are retained in a given time or suspension volume. The outlet particle flux or outlet particle density could also take a number of forms corresponding to those discussed above in respect of the particle clearance rate.

The particles monitored in accordance with the invention will typically be food particles such as plankton. It is even envisaged that the measurements could identify particles of different types and either exclude some particles from the calculations (e.g. particles of non-food debris) or attach different weightings to different particles in the calculations. These different weightings could be based on many factors or combination of factors such e.g. the food value.

The Applicant has recognised that it is not always essential to measure actual particle fluxes on either side of the organism. In some circumstances it may be sufficient to make assumptions about the density of food particles in the surrounding water in which case the apparatus could be arranged simply to measure the pumping rate of the organism, that is the rate at which ambient water passes through it. The assumed density of food particles could be measured periodically or it may even be assumed to remain relatively constant such that pumping rate is just used a proxy for clearance rate.

Thus in a set of embodiments, the first parameter is a pumping rate. In some preferred embodiments, the method comprises the steps of:
performing a series of first measurements, of a flow of water in a region adjacent a feeding orifice of said organism;
performing a series of second measurements, of a flow of water in a region adjacent an excretion orifice of said organism; and
using said first and second measurements to calculate a pumping rate for said organism.

Whilst it is possible in accordance with the foregoing embodiments to measure volume flow rates directly—i.e. the volume of particles or volume of water passing per unit time—this is not essential. In a set of embodiments an estimate of the volume flow rate is achieved by measuring the flow speed of the water. For a given siphon cross-section which the organism presents, this will be proportional to the volume flow rate. Since in accordance with at least some embodiments of the invention only changes in the particle clearance rate are important, this may be sufficient. Alternatively the cross-sectional area may be estimated or measured to yield an estimate of the actual volume flow rate.

The Applicant has appreciated that in practice the siphon cross-sectional area of the organism will depend on the degree of gaping—i.e. whether the mussel or other organism is fully open. It can either be assumed that this is the case (or at least that this will be the case over a measurement cycle and therefore that a maximum flow speed should be used) or the degree of gaping may be measured. One way of doing this is described in “A fiber optic sensor for high resolution measurement and continuous monitoring of valve gape in bivalve molluscs” Journal of Shellfisheries Research, August, 2007 by Dana M. Frank, John F. Hamilton, J. Evan Ward, Sandra E. Shumway.

Thus the flow rates from which the pumping rate is calculated could be measured in a variety of ways—e.g. with a separate sensor, but in a set of embodiments the pumping rate is determined using the velocity vectors of particles carried in the water. In one possible set of embodiments a set of sensors could be used continuously or frequently to measure water flow speed, which inherently requires relatively less processing power, and periodically or less frequently also to determine particle density, which requires relatively greater processing. Water flow rate (via flow speed) could for example be measured locally with particle density being calculated from analysis carried out remotely.

Measurements of particle density or flux may be carried out in a number of different ways. For example, one or more bulk properties of the volume of water could be measured such as the transmissivity, reflectivity or absorbance of the water to light (i.e. the optical density of the water), other electromagnetic radiation or sound; combined with knowledge obtained either theoretically or empirically as to how the density or flux influences sensors. Alternatively the change in frequency distribution of a signal emitted into the water after transmission or reflection through the volume being measured could be determined.

Thus in some embodiments, the first parameter is an optical density of the water in a region adjacent an excretion orifice of the organism. The optical density may be expressed as a percentage or fraction of the optical density of ambient water or of water in a region adjacent a feeding orifice of the organism.

In a set of preferred embodiments however, measurements of individual particles are carried out. There are again a number of techniques which could be used. The water could be imaged and image processing techniques used to locate individual particles. In some preferred embodiments a laser particle detector is employed. Suitable examples include a miniature laser Doppler velocimeter (mini-LDV), laser Doppler anemometer (LDA), particle image velocimeter (PIV), or a time-of-flight velocity sensor. Suitable sensors are available commercially from Measurement Science Enterprise, Inc. of Pasadena, Calif. or Dantec Dynamics of Skovlunde, Denmark. These sensors can of course carry out simple water flow speed measurements referred to above either in addition to or instead of particle density/flux measurements.

Sessile organisms suitable for use in the invention include filter feeders, ascidians sponges and bivalves such as mussels, scallops, clams, etc.

Although the method herein described may be performed on a single organism, it is preferred that this is carried out simultaneously or sequentially on a plurality of organisms from the same species. In this way, accuracy of the monitoring methods may be improved by measuring the clearance rate, outlet particle flux, outlet particle density or any other pollution-sensitive parameter of a statistically significant sample. In such embodiments a common radiation source and/or detector are preferably employed. For example the method could comprise bringing a radiation-and-detector arrangement into successive mutual alignment with each of a plurality of organisms.

In an exemplary embodiment, a plurality of sessile organisms may be distributed around the rim of a disc or the outer surface of a cylinder. Each may be associated with its own detector, or one or more detectors may be shared between a greater number of organisms, in which case a mechanical arrangement could be employed to move the sensor or organism into mutual proximity for conducting measurements. In a set of embodiments, the method comprises the use of one or more detectors arranged to measure the particle flux or density in the common environment of all the organisms, from which they draw in their food, whereas each organism is provided with its own detector to detect its exhaled particle flux or density or flow speed.

The Applicant has appreciated that for algal particles, one way of measuring particle flux in flow to measure clearance rate is to measure the concentration of chlorophyll a in the region of the feeding and excretion orifices respectively. This can be done by measuring chlorophyll a fluorescence.

Thus in some embodiments, the method comprises the steps of:
performing a series of first measurements, of chlorophyll a fluorescence in said aquatic mass in a region adjacent a feeding orifice of said organism;
performing a series of second measurements, of chlorophyll a fluorescence in said aquatic mass in a region adjacent an excretion orifice of said organism; and
using said first and second measurements to calculate a particle clearance rate for said organism.

Alternatively, the Applicant has further appreciated that outlet particle flux alone might be measured using chlorophyll a fluorescence (i.e. chlorophyll a fluorescence in a region adjacent an excretion orifice of the organism). Thus, in a set of embodiments, the first parameter of the invention is chlorophyll a fluorescence measured in a region adjacent an excretion orifice of the organism.

As mentioned above, an alternative to clearance rate is oxygen consumption of the organism. The oxygen consumption of an organism is the difference between the oxygen content of its water intake and the oxygen content of its water outflow which can be measured to infer metabolism. This can be used for environmental monitoring of pollution effect of the type taught herein since metabolic rate exhibits an reversed relationship with pollution.

Thus in a set of embodiments, the first parameter is an oxygen consumption rate. In some embodiments, the method comprises the steps of:
performing a series of first measurements, of oxygen concentration in said aquatic mass in a region adjacent a feeding orifice of said organism;
performing a series of second measurements, of oxygen concentration in said aquatic mass in a region adjacent an excretion orifice of said organism; and
using said first and second measurements to calculate an oxygen consumption rate for said organism.

As an alternative the oxygen concentration in a region adjacent an excretion orifice alone can be used to infer metabolic rate and thus can be used as an indicator of pollution level.

Thus, in another set of embodiments, the first parameter is oxygen concentration measured in a region adjacent an excretion orifice of the organism.

In another preferred set of embodiments, the first parameter is a growth rate.

In some preferred embodiments the method comprises the use of a sessile organism exhibiting apical growth, an edge so as to form a gap between it and a tip of the sessile organism, an electromagnetic source arranged such that a beam therefrom impinges upon said gap to produce a diffraction pattern, and a corresponding electromagnetic detector arranged to detect said diffraction pattern and means for monitoring a change in said diffraction pattern over time which is indicative of the natural growth of the apical tip of said organism.

In a set of embodiments at least one further sensor is employed in association with the or each organism in order to detect a different pollution-dependent characteristic thereof. Such embodiments are beneficial as they exploit the dependence of two parameters on the level of environmental pollution. For example, these two parameters could be clearance rate/pumping rate and growth rate, or clearance rate/pumping rate and oxygen consumption rate, although it will be appreciated that other combinations of pollution-dependent parameters may be used. This can allow more accurate indications to be given either where an indication from one parameter can act as validation of the other, or where the two parameters have different (albeit likely overlapping) sensitivities in terms of pollutant substances, concentration sensitivity, or response time-scales.

Thus in a preferred set of embodiments, the method is employed twice, wherein the first time the method is employed, the first parameter is a first pollution-sensitive parameter and the second time the method is employed, the first parameter is a second pollution-sensitive parameter. The first and second pollution-sensitive parameters are selected from the group comprising: particle clearance rate, pumping rate, growth rate, oxygen consumption rate, outlet particle flux, outlet particle density, outlet chlorophyll a fluorescence, outlet oxygen concentration and optical density of outlet water (where outlet indicates that the parameter is measured in a region adjacent an excretion orifice of an organism). Outlet particle flux, outlet particle density, outlet chlorophyll a fluorescence, outlet oxygen concentration and optical density of outlet water may be expressed as a percentage or fraction of the corresponding value in ambient water or of the corresponding value adjacent a feeding orifice of the organism.

The Applicant has appreciated that combining two pollution-sensitive parameters for which the presence of pollution causes the envelope of one parameter to change in the opposite manner from the envelope of the other (i.e. one envelope increases while the other decreases) provides a more sensitive indicator of the presence of pollution. For example, oxygen consumption rate combined with particle clearance rate, pumping rate or growth rate, or as another example, outlet oxygen concentration combined with outlet particle flux, outlet particle density, outlet chlorophyll a fluorescence or optical density of outlet water.

Thus, in some embodiments in which the method of the invention is employed twice, the first and second pollution-sensitive parameters are selected such that the envelope associated with one of the pollution-sensitive parameters increases with increasing pollution level and the envelope associated with the other pollution-sensitive parameter decreases with increasing pollution level.

Which sessile organisms are suitable for use in a given embodiment of the invention depends on the parameters being measured. As mentioned previously, those suitable for measuring clearance rate include filter feeders, especially bivalves, such as mussels, scallops, clams, etc. These bivalves are also suitable for measuring apical growth, typically measured at the apex (lip) of the shell and are thus particularly preferred for use where these two measurements are to be carried out. Preferably the sessile organism of which growth is to be measured is a young individual in the growth phase.

In accordance with all aspects of the invention it is preferred that data relating to individual organisms is analysed separately from that relating to other individual organisms. This stems from the recognition that the change in the behaviour of any given individual is the most sensitive indicator of the effect of pollution, rather than changes in aggregated or averaged behaviour of a plurality of organisms.

Embodiments of the invention will now be described with reference to the accompanying drawings, in which:

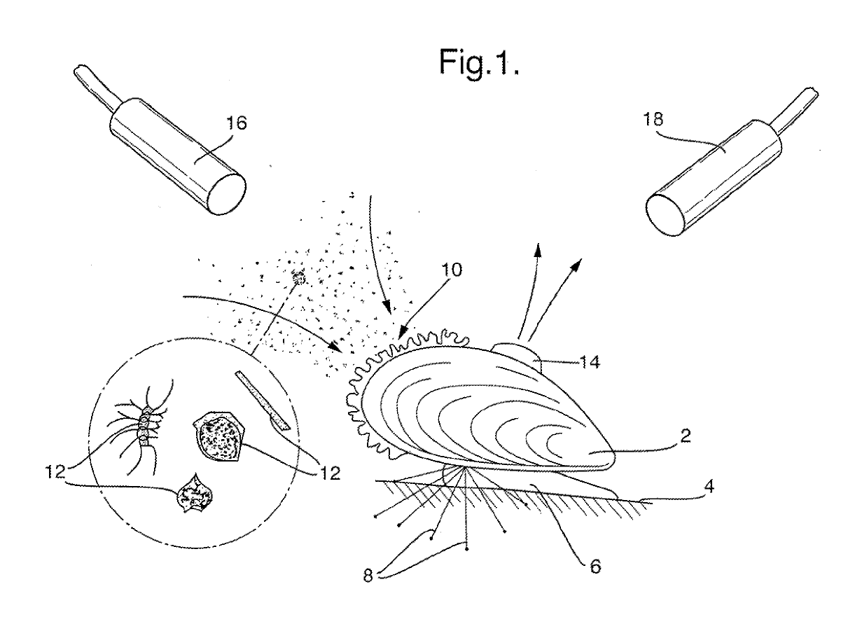

FIG. 1 is a schematic view illustrating the principle of measurement of the clearance rate of a mussel;

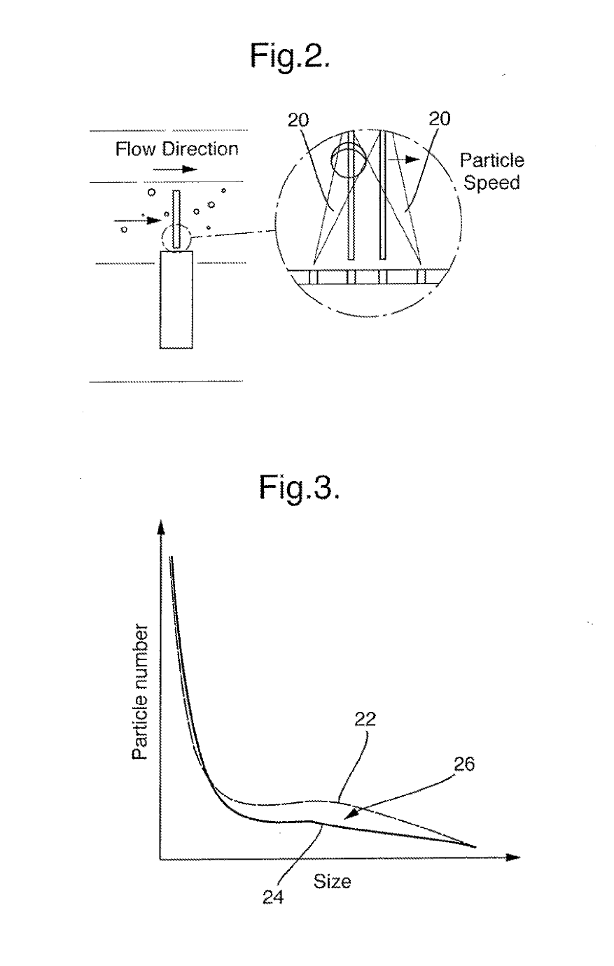

FIG. 2 is a diagram illustrating the principle on which the detectors of one particular embodiment operate;

FIG. 3 is a graph showing how food uptake rate is calculated;

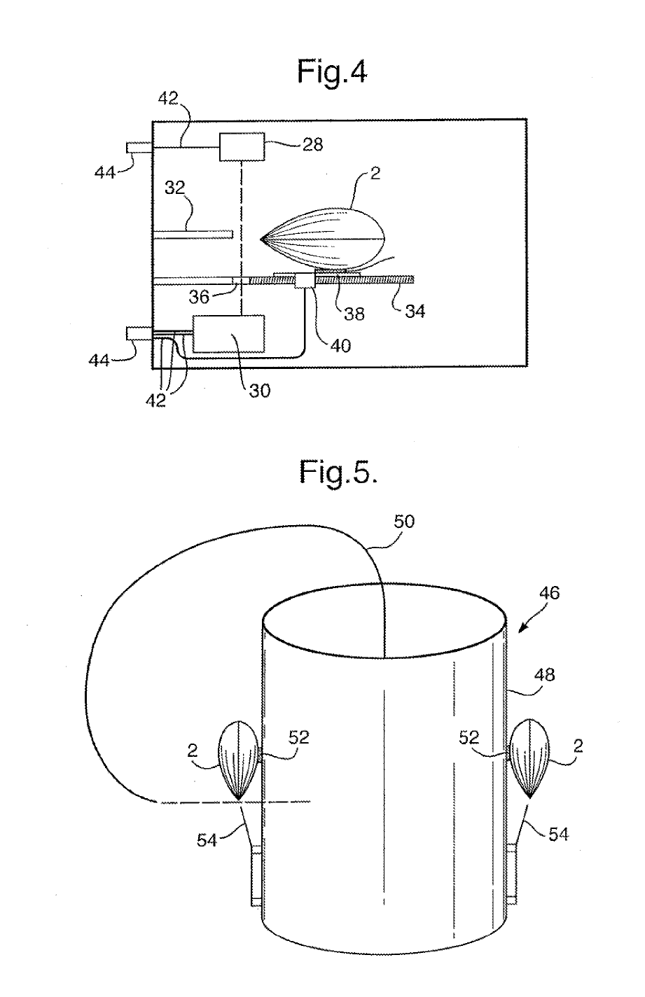

FIG. 4 is a schematic drawing of part of a biosensor unit according to an embodiment of the invention showing how apical growth can also be measured;

FIG. 5 is a schematic drawing of a biosensor unit which is adapted to simultaneously monitor the clearance rate and growth rate of a plurality of sessile organisms;

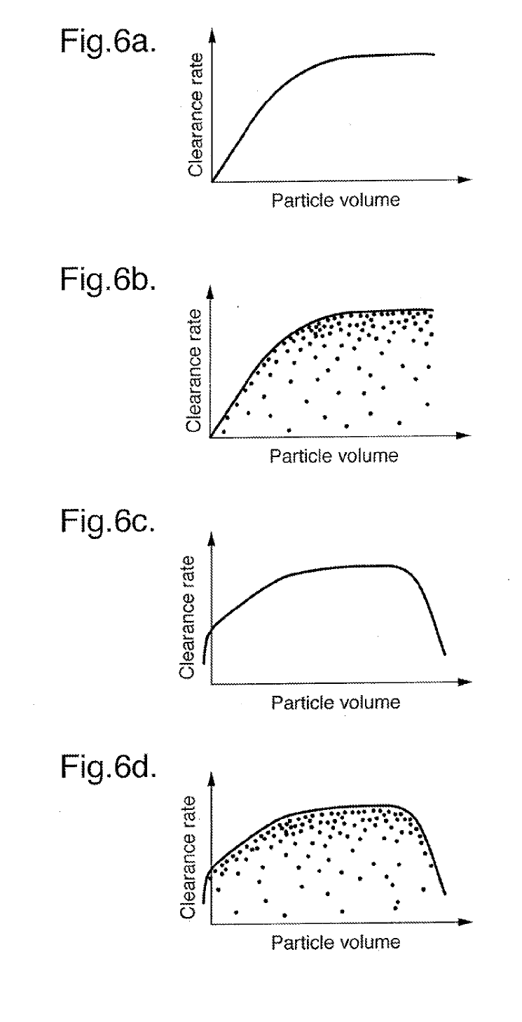

FIGS. 6a and 6b are plots showing respectively theoretical and measured relationships between clearance rate and particle volume;

FIGS. 6c and 6d are plots showing respectively theoretical and measured relationships between clearance rate and temperature;

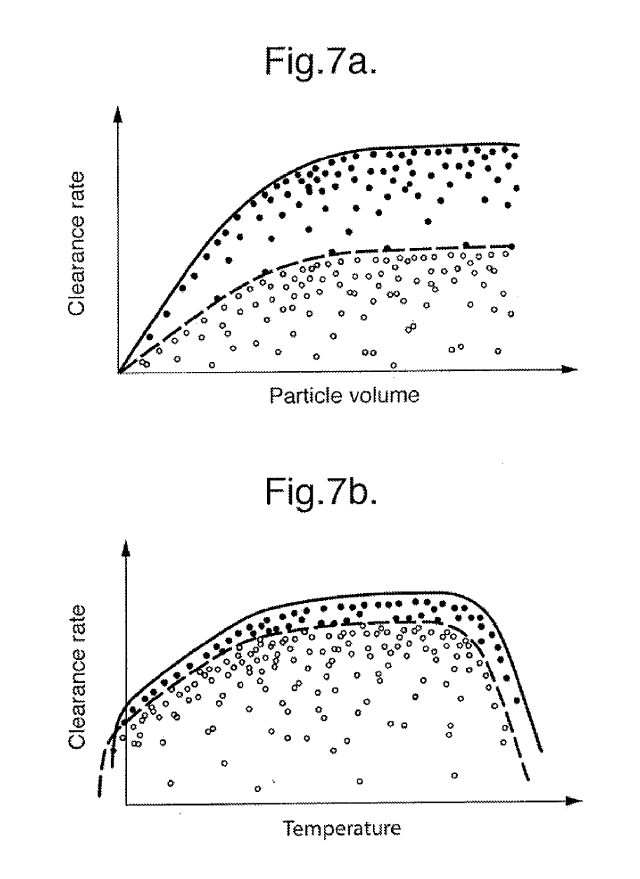

FIGS. 7a and 7b are plots of particle clearance rate against particle volume and temperature respectively showing the effect of pollution;

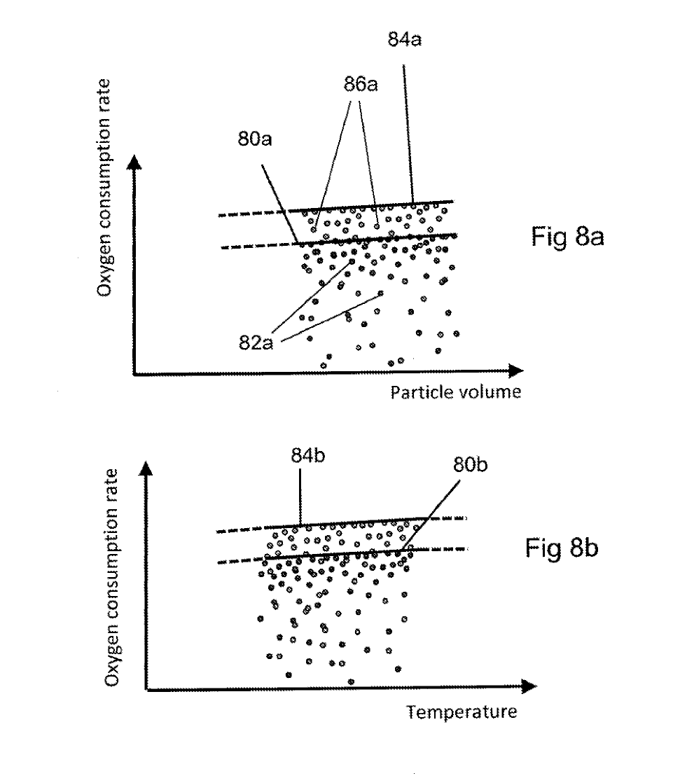

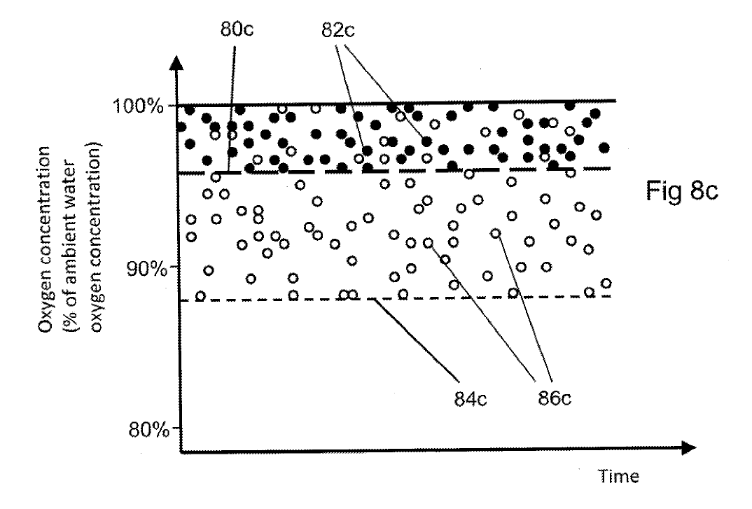

FIGS. 8a and 8b are plots of oxygen consumption rate against particle volume and temperature respectively showing the effect of pollution;

FIG. 8c is a plot of outlet oxygen concentration (as a percentage of oxygen concentration of ambient water) against time showing the effect of pollution; and

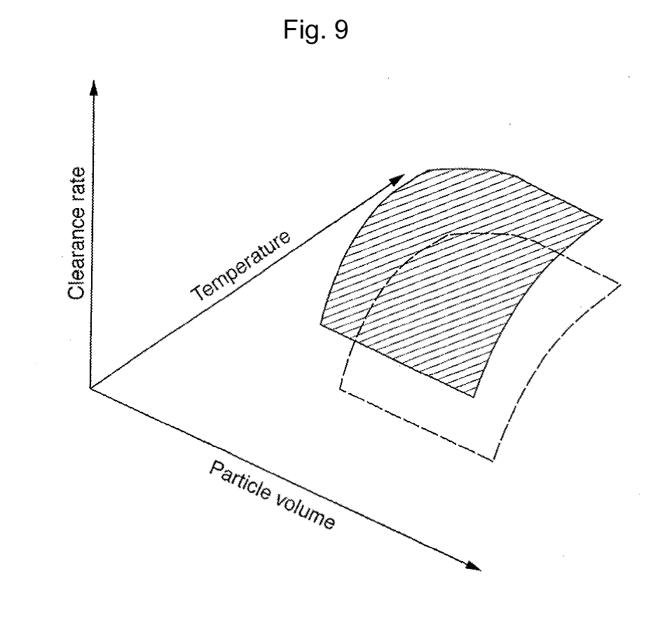

FIG. 9 is a three-dimensional plot of particle clearance rate against particle volume and temperature showing the effect of pollution.

FIG. 1 shows a mussel 2 which is a filter feeder bivalve sessile organism. It is attached to a surface 4 by means of its foot 6 and byssus threads 8. At the rounded end of the mussel 2 is an inhalant siphon 10 which sucks in water in which particles including plankton 12 are suspended. The mussel 2 has an exhalant siphon 14 along its upper edge.

Two particle flux sensors 16, 18 are disposed in the vicinity of the inhalant siphon 10 and the exhalant siphon 14 respectively. Many different types of sensors could be used but in one example they are ‘micro-V’ sensors available from Measurement Science Enterprise, Inc. of Pasadena, Calif. However neither the type of sensor nor the manufacturer is essential. As illustrated in FIG. 2, these particular sensors operate by measuring the respective reflections of a pair of spaced laser beams 20 from a particle passing through a particular volume of water. The reflections can be used to estimate the size of the particle, and also its velocity using its time of flight between the laser beams. The individual sensors 16, 18 can therefore, in addition to flow, also measure the number and size distribution of particles passing through a given volume and therefore the average particle flux. Other sensors work in different ways. For example a simple measurement of flow speed may be made in order to infer the particle flux.

In other embodiments fluorescence sensors measuring chlorophyll a concentration may be employed to estimate algal particle flux and thus calculate clearance rate.

Additionally or alternatively micro-sensors for oxygen could be used. Such sensors detect the change in oxygen between in-flowing water and out-flowing water. This gives a measure of the organism's metabolic rate at any given time. Metabolic rate and thus oxygen use tends to increase in the presence of increasing pollution

Oxygen micro-sensors and fluorescence sensors can be used alone or together, especially in water containing sand or clay particles. The sensors may be placed in the inflow/outflow currents or may be focussed on them, depending on the type of sensor.

FIG. 3 shows two plots 22, 24. The upper plot 22 corresponds to the size distribution of particles measured by the inhalant sensor 16 and the lower plot 24 corresponds to the size distribution of particles measured by the exhalant sensor 18. These plots can be generated by integrating the measured flux over a given time.

The area between the plots 26 represents the food uptake rate or clearance rate of the mussel 2. By measuring the food uptake rate, the scope for growth of the mussel can be estimated relatively accurately. Thus successive measurements for a given individual organism can be used to indicate changes in the scope for growth which can indicate changing levels of aquatic pollution. The sensors 16, 18 may transmit raw data to a remote computer for processing and analysis or the data may be stored and/or processed locally in the submersed biosensor unit.

FIG. 4 shows, schematically, how apical growth of the mussel 2 can also be measured.

Within the biosensor unit are disposed a He—Ne laser source 28, CCD light detector 30 and a blade 32. The laser source 28 is arranged such that the beam it generates is aligned to pass immediately adjacent the edge of blade 32.

Also within biosensor unit 1 are disposed two parallel threaded tracks 34 carrying between them a fixed beam stop 36 and a mobile carriage 10. The carriage 10 is attached to a drive motor 40 operation of which causes the carriage 10 to move towards or away from the blade 32. The carriage 10 is provided with position location means (not shown) which provide a data signal indicating the relative spacing between the carriage 10 and the blade 32. The mussel 2 is mounted on the carriage 10 with the growing edge of the shell tip pointing towards the blade 32. The laser source 28, light detector 30 and drive motor 40 are provided with power and data transmission leads 42 to connectors 44.

In operation, the motor 40 is engaged to draw the mussel 2 towards the blade 32 until a predetermined light diffraction pattern (i.e. one having readily discernible dark and light spots) is detected by the detector 30. The average distance (d) between adjacent spots in the diffraction pattern is inversely proportional to the width of the slit (a), which can be calculated from the formula a=λ·s/d, where s is the vertical distance from the slit to the diffraction pattern and λ is the wavelength of the laser light.

After a set period of time (e.g. 24 hours), a further diffraction pattern is detected by the detector 30 and used to calculate the width of the slit. The reduction in slit width provides an indication of the apical growth of the mussel 2. Such measurements will generally be repeated over a period of several days (or, as appropriate, several months) until the apical tip of the mussel 2 is almost touching the blade 32 (i.e. when the diffraction pattern is almost diminished). At that point, the motor 40 is engaged to draw the mussel away from the blade 32 until an optimum diffraction pattern is once again obtained. The process may then be repeated to further monitor the growth rate of the mussel 2.

In an exemplary application of the embodiment set out above, the apparatus is set up to measure diffraction patterns over a range of slit apertures of 100-900 μm but to re-enlarge the aperture by moving the mussel carrier when the slit has been reduced to 200 μm. Under normal conditions in the summer with ample food (algae) in the in the water typical shell growth is of the order of 50 μm/day although can be as high as 100 μm/day. The aperture is typically re-enlarged on a weekly basis. In winter shell growth can be less than 1 μm/day and so adjustment needs only to be made at two-monthly intervals.

Referring to FIG. 5 there is shown a biosensor unit 46 in accordance with an embodiment of the invention. In this embodiment the biosensor unit comprises a transparent water impervious cylinder 48 having disposed therein various sensor units (not shown). These include particle flux sensors such as those shown in FIG. 1 and a light detector such as that shown in FIG. 4. The biosensor unit 46 also includes an optical fibre 50 connected to a source of laser light (not shown) positioned within the cylinder 48.

Mounted on the outer surface of the cylinder 46, using adhesive 52, are a plurality of mussels 2 and a plurality of plastic tabs 54. Each mussel 2 is mounted such that the growing edge of its shell tip is pointing towards a plastic tab 54. The optical fibre 50 and detector may be manipulated such that the laser beam is aligned to impinge on the gap between the edge of the plastic tab 54 and the tip of the mussel. The corresponding diffraction pattern is recorded. The laser-detector arrangement is then indexed round to the next organism to measure that gap. Of course the organisms could be moved (by rotating the cylinder) or each organism could be provided with its own detector.

Similarly a plurality of particle flux sensors is provided—one for each mussel 2 in the vicinity of its exhalant siphon. One or more further particle flux sensors is provided to determine the particle density in the common environment of the mussels 2 in order to establish the intake size distribution.

The mussels 2 are used individually to provide measurements of the effects of pollution by measuring growth and particle clearance rate for each one individually. In any given individual the growth and clearance rate will be altered from their established background levels (which may differ from one mussel to the next) in the presence of pollution. Thus by measuring the effect on individual mussels, an indication of pollution can be obtained from each.

FIG. 6a shows schematically the theoretical relationship between particle clearance rate and particle volume for a particular organism—e.g. a mussel. FIG. 6b shows a representative plot of various clearance rate and particle volume data points 60 as might be actually measured. The scatter is partly accounted for by short-term opening and closing of the mussel; as the mussel closes, its clearance rate is diminished. The line 62 at the top represents the upper envelope of the data points 60 in respect of particle clearance rate. Clearly as more data points are measured, the more clearly this envelope will emerge. The envelope 62 approximates the theoretical relationship shown in FIG. 6a. The envelope 62 corresponds to the mussel being fully open.

FIG. 6c shows a schematic theoretical relationship between temperature and particle clearance rate. Again as actual data points 64 are measured, an envelope curve 66 will emerge as shown in FIG. 6d.

FIG. 7a shows the plot of FIG. 6b but with a second set of particle clearance rate/particle volume data points 68 (shown in lighter shading) superimposed representing measurements taken at a later time than the first set. It will be seen that the second set of data points has a lower envelope curve 70. This is a strong indicator that the individual organism to which the data relate has been exposed to aquatic pollution. Thus by measuring the shift in the envelope curve, the presence or increase in pollution between the time of the first measurement set and the time of the second measurement set can be detected.

FIG. 7b shows that the particle clearance rate/temperature envelope curve exhibits a similar relationship in that it shifts downwards from the initial position 66 corresponding to the first measurements, to the later position 72 corresponding to the second set of measurements taken in the presence of pollution.

FIG. 8a shows a representative plot of a first set of various oxygen consumption rate and particle volume data points 82a for a first time period and a second set of data points 86a for a second time period, as might be actually measured. Upper envelopes 80a and 84a are visible for the first and second data sets respectively. The envelope 84a corresponding to the second data set, which represents measurements taken in the presence of pollution, is higher than the envelope 80a corresponding to the first data set due to the increase in oxygen consumption of the organism in the presence of pollution.

FIG. 8b shows a shift upwards of the oxygen consumption rate/temperature upper envelope curve from the initial position 80b corresponding to the first measurements, to the later position 84b corresponding to the second set of measurements taken in the presence of pollution.

FIG. 8c shows a representative plot of a first set of data points 82c of outlet oxygen concentration against time for a first time period and a second set of data points 86c for a second time period, as might be actually measured. The scatter is partly accounted for by short-term opening and closing of the organism which causes mixing of the outflow water of the organism with ambient water. In the case of oxygen concentration, as more data points are measured, a lower envelope 80c emerges for the first set of data points 82c, and different lower envelope 84c emerges for the second set of data points 86c. The second lower envelope 84c is lower than the first lower envelope 80c, indicating an increased level of pollution during the second time period.

FIG. 9 shows that the dependence of the particle clearance rate on particle volume and on temperature can be represented simultaneously using a three-dimensional plot. Here the data envelope is a surface which moves from an initial position 74 before pollution is introduced to a later position 76 after the introduction of pollution. Combining both parameters in this way can give a more reliable indicator.

At any point x-y point on this plot (i.e. for any combination of particle volume and temperature) a pollution effect index can be defined as the ratio of the heights of the two envelope surfaces in the corresponding vertical column.

Thus the pollution effect index based on clearance rate PI_{CL}=CLₘₐₓ/CLₛᵤₚₚ

where:
CLₘₐₓ is the maximum clearance rate (represented by the upper surface 74); and
CLₛᵤₚₚ is the suppressed clearance rate (represented by the lower surface 76).

The value of PI_{CL} may differ across the x-y plane but such variations are likely to be minimal over relatively small sections of the surfaces and under stable conditions. If necessary an average over a certain area could be taken.

Although this aspect of the invention is illustrated above using particle clearance rate (CL), similar relationships are observed for pumping rate (V) and growth rate (U). Thus in an analogous manner pollution indices may be defined:

PI_{V}=Vₘₐₓ/Vₛᵤₚₚ

PI_{U}=Uₘₐₓ/Uₛᵤₚₚ

A similar index can be defined for oxygen consumption although this will be inverted compared to those corresponding to particle clearance and growth rates due to the relative inverse dependency of oxygen consumption on pollution—i.e. as pollution increases so does oxygen consumption (OC). Thus in an analogous manner pollution indices may be defined:

PI_{OC}=OCₘᵢₙ/OCₛᵤₚₚ

where PI_{OC} decreases with increased pollution.

Different indices may be more sensitive to different types of pollution and thus one or more of these indices could be monitored as part of an aquatic pollution monitoring system. The Applicant has appreciated that combining any of the other indices with the one of oxygen consumption will give a new more sensitive index as the other indices move in the opposite direction (i.e. they increase rather than decrease) in response to an increased level of pollution.

1. A method of monitoring the effect of pollution in an aquatic mass comprising the steps of:
placing a living sessile organism in said aquatic mass;
carrying out a series of first measurements of a first parameter of said organism during a first time period;
recording each of said first measurements with an associated measurement of a second parameter to give a series of first data points;
determining and recording a first envelope with respect to said first parameter for said first data points;
carrying out a series of second measurements of said first parameter of said organism during a second time period;
recording each of said second measurements with an associated measurement of a second parameter to give a series of second data points;
determining and recording a second envelope with respect to said first parameter for said second data points; and
comparing said first and second envelopes to determine the presence of pollution during said first or second time period;
wherein the first and second envelopes are lower envelopes.

2. (canceled)

3. The method of claim 1 wherein the first parameter is a particle clearance rate.

4. The method of claim 1 wherein the first parameter is a pumping rate.

5. The method of claim 1 wherein the first parameter is a growth rate.

6. The method of claim 1 wherein the first parameter is an oxygen consumption rate.

7. The method of claim 1 wherein the first parameter is a particle flux in a region adjacent an excretion orifice of the organism.

8. The method of claim 1 wherein the first parameter is a particle density in a region adjacent an excretion orifice of the organism.

9. The method of claim 1 wherein the first parameter is an optical density of water in a region adjacent an excretion orifice of the organism.

10. The method of claim 1 wherein the first parameter is a chlorophyll a fluorescence in a region adjacent an excretion orifice of the organism.

11. The method of claim 3 comprising:
performing a series of first measurements of a flux of particles in said aquatic mass in a region adjacent a feeding orifice of said organism;
performing a series of second measurements of a flux of particles in said aquatic mass in a region adjacent an excretion orifice of said organism; and
using said first and second measurements to calculate the particle clearance rate for said organism.

12. The method of claim 7 comprising measuring a particle size distribution.

13. The method of claim 7 comprising measuring the pumping rate of the organism.

14. The method of claim 4 comprising:
performing a series of first measurements of a flow of water in a region adjacent a feeding orifice of said organism;
performing a series of second measurements of a flow of water in a region adjacent an excretion orifice of said organism; and
using said first and second measurements to calculate the pumping rate for said organism.

15. The method of claim 13 comprising determining the pumping rate using velocity vectors of particles carried in the water.

16. The method of claim 11 comprising measuring a water flow speed.

17. The method of claim 16 comprising measuring a gape of the organism.

18. The method of claim 11 comprising using a set of sensors continuously or frequently to measure water flow speed and periodically or less frequently also to determine particle density.

19. The method of claim 11 comprising measuring individual particles.

20. The method of claim 19 comprising using a laser particle detector.

21. The method of claim 1 comprising measuring said first parameter individually for each of a plurality of organisms.

22. The method of claim 7 comprising measuring a particle flux or density in a common environment of the organisms.

23. The method of claim 11 comprising measuring chlorophyll a fluorescence in the region of the feeding and excretion orifices of the organism respectively.

24. The method of claim 3 comprising:
performing a series of first measurements of chlorophyll a fluorescence in said aquatic mass in a region adjacent a feeding orifice of said organism;
performing a series of second measurements of chlorophyll a fluorescence in said aquatic mass in a region adjacent an excretion orifice of said organism; and
using said first and second measurements to calculate the particle clearance rate for said organism.

25. The method of claim 7 comprising using at least one further sensor in association with the or each organism in order to detect a different pollution-dependent characteristic thereof.

26. The method of claim 6, comprising:
performing a series of first measurements of oxygen concentration in said aquatic mass in a region adjacent a feeding orifice of said organism;
performing a series of second measurements of oxygen concentration in said aquatic mass in a region adjacent an excretion orifice of said organism; and
using said first and second measurements to calculate the oxygen consumption rate for said organism.

27. The method of claim 1 wherein the first parameter is an oxygen concentration in a region adjacent an excretion orifice of the organism.

28. The method of claim 7 wherein said organism is a sessile organism exhibiting apical growth, said method comprising measuring said apical growth.

29. The method of claim 1 wherein the first parameter is a first pollution-sensitive parameter;
wherein the second parameter is a second pollution-sensitive parameter;
wherein the first and second pollution-sensitive parameters are selected from the group comprising particle clearance rate, pumping rate, growth rate, oxygen consumption rate, outlet particle flux, outlet particle density, outlet chlorophyll a fluorescence, outlet oxygen concentration and optical density of outlet water.

30. The method of claim 29 wherein the first and second pollution-sensitive parameters are selected such that the envelope associated with one of the first and second pollution-sensitive parameters increases with increasing pollution level and the envelope associated with the other pollution-sensitive parameter decreases with increasing pollution level.

31. A non-transitory computer-readable medium comprising instructions for configuring a processor to carry out steps of:
receiving an input of a series of first measurements of a first parameter of an organism during a first time period;
receiving an input of an associated measurement of a second parameter for each of said first measurements;
recording each of said first measurements with the associated measurement of the second parameter to give a series of first data points;
determining and recording a first envelope with respect to said first parameter for said first data points;
receiving an input of a series of second measurements of said first parameter of said organism during a second time period;
receiving an input of an associated measurement of the second parameter for each of said first measurements;
recording each of said second measurements with the associated measurement of the second parameter to give a series of second data points;
determining and recording a second envelope with respect to said first parameter for said second data points; and
comparing said first and second envelopes to determine the presence of pollution during said first or second time period.
